SEQUENCE LISTING (19 sequences)

SEQ ID NO: 1 - protein, 25 aa, Homo sapiens
LDKWLLPRNPPSEDLPGPGGSVDIV

SEQ ID NO: 2 - protein, 21 aa, Homo sapiens
CSSALASKPSPEGASAASFEY

SEQ ID NO: 3 - protein, 22 aa, Homo sapiens
GGLSDGPYSNPYENSLIPAAEP

SEQ ID NO: 4 - protein, 5 aa, Mus musculus
RLFAY

SEQ ID NO: 5 - protein, 17 aa, Mus musculus
TISGRGTYTYYPDSVKG

SEQ ID NO: 6 - protein, 10 aa, Mus musculus
GFTFSSYAMS

SEQ ID NO: 7 - protein, 9 aa, Mus musculus
CQGTHFPYT

SEQ ID NO: 8 - protein, 7 aa, Mus musculus
LVSHLDS

SEQ ID NO: 9 - protein, 16 aa, Mus musculus
KSSQSLLDSDGKTYLN

SEQ ID NO: 10 - protein, 114 aa, Mus musculus
AVQVVESGGDLVKPGGSLQLSCAASGFTFSSYAMSWARQTPEKRLEWVATISGRGTYTYYPDSVKGRFTISRDNAKNILYLQMSSLRSEDTAMYYCTPRLFAYWGQGTLVTVSA

SEQ ID NO: 11 - protein, 112 aa, Mus musculus
DVVMTQTPLTLSVTIGEPASISCKSSQSLLDSDGKTYLNWFLQRPGQSPKRLIYLVSHLDSGVPDRFTGSGSGTDFTLKISRVEAEDLGVYYCCQGTHFPYTFGGGTDLEIK

SEQ ID NO: 12 - protein, 10 aa, Mus musculus
LMVPTYGLDY

SEQ ID NO: 13 - protein, 17 aa, Mus musculus
QIYPGDGDTNYIGDFKG

SEQ ID NO: 14 - protein, 10 aa, Mus musculus
GYTFSRYWMN

SEQ ID NO: 15 - protein, 9 aa, Mus musculus
KESYILPYT

SEQ ID NO: 16 - protein, 7 aa, Mus musculus
WASTRES

SEQ ID NO: 17 - protein, 17 aa, Mus musculus
KSSQSLLNSRTRKNYLA

SEQ ID NO: 18 - protein, 119 aa, Mus musculus
QVQLQQSGAELVRPGSSVKISCKASGYTFSRYWMNWVKQRPGQGLEWIGQIYPGDGDTNYIGDFKGKATLTADKSSSIAYMQLSSLTSEDSAVYFCARLMVPTYGLDYWGQGTSVTVSS

SEQ ID NO: 19 - protein, 113 aa, Mus musculus
DIVMSQSPSSLAVSAGEKVTMSCKSSQSLLNSRTRKNYLAWYQQKPGQSPKLLIYWASTRESGVPDRFTGSGSGTDFTLTIYSVQAEDLAVYYCKESYILPYTLGGGTKLEIK

(19) United States
(12) Patent Application Publication
(10) Pub. No.: US 2011/0143372 A1
(43) Pub. Date: Jun. 16, 2011

(54) ANTIBODIES AGAINST HUMAN EPO RECEPTOR

(75) Inventors: Michael Jarsch, Bad Heilbrunn (DE); Manfred Kubbies, Penzberg (DE); Olaf Mundigl, Weilheim (DE); Nora Torres-Nagel, Habach (DE)

(73) Assignee: Hoffman-La Roche, Inc., Nutley, NJ (US)

(21) Appl. No.: 13/035,762

(22) Filed: Feb. 25, 2011

(30) Foreign Application Priority Data
Aug. 28, 2008 (EP) 08015178.0
Jan. 15, 2009 (EP) 09000500.0
Feb. 13, 2009 (EP) 09002001.7

(51) Int. Cl. G01N 33/567; C07K 16/28

(52) U.S. Cl. 435/7.21; 530/387.9; 435/7.1

(57) ABSTRACT

An antibody binding to human EPO receptor, characterized in specifically binding EPO receptor fragment LDKWLLPRNPPSEDLPGPGGSVDIV (SEQ ID NO:1), CSSALASKPSPEGASAASFEY (SEQ ID NO:2), or GGLSDGPYSNPYENSLIPAAEP (SEQ ID NO:3) is useful for the analysis of EPO receptor in human tissue.

### CROSS REFERENCE TO RELATED APPLICATIONS

This application is a 371 of International Application No. PCT/EP2009/006174, filed 26 Aug. 2009, and claims the benefit of priority under 35 USC §119(a) to European patent application number 08015178.0, filed 28 Aug. 2008, European patent application number 09000500.0, filed 15 Jan. 2009, and European patent application number 09002001.7, filed 13 Feb. 2009, the disclosures of which are incorporated herein by reference in their entirety.

### BACKGROUND OF THE INVENTION

Human erythropoietin (EPO) is a 166-aa glycoprotein which is involved in the proliferation and differentiation of erythroid progenitor cells. These cellular responses are mediated by the human EPO receptor (EPO receptor, EPO-R), a 508-aa glycoprotein. EPO-R is a protein of 508 amino acid length (Swiss Prot P19235) containing a single transmembrane domain and has been classified as a member of the growth hormone subfamily of class I cytokine receptors. EPO-R is described, e.g., in Winkelmann, J. C., et al., Blood 76 (1990) 24-30, and Jones, S. S., et al., Blood 76 (1990) 31-35).

Antibodies against EPO-R are known from, e.g., D'Andrea, A. D., Blood 82 (1993) 46-52; Elliott, S., Blood 107 (2006) 1892-1895; Kirkeby, A., J. Nerosci. 164 (2007) 50-58; Miura, O., Arch. Biochem. 306 (1993) 200-208; and EP1 146 056, EP 1 327 681, EP 0 773 962, EP 0 776 370, US 2002/0031806, US 2003/0215444, US 2004/0058393, US 2004/0071694, US 2004/0175379, US 2005/0227289, US 2005/0244409, US 2006/0018902, U.S. Pat. No. 6,998,124, U.S. Pat. No. 7,053,184, U.S. Pat. No. 7,081,523, WO 1995/005469, WO 1996/003438, WO 2000/061637, WO 2004/035603 A2, WO 2005/100403 A2. However, studies investigating the expression and localization of EPOR in tissue samples produce divergent and often artifactual results because lack of specificity of known antibodies against EPO-R (see Jelkmann, W., et al., Crit. Rev. Onc/Hematol. 67 (2008) 39-61; Elliott, S., et al., Blood 107 (2006) 1892-1895; Jelkmann, W. and Laugsch, M., J. Clin. Oncol. 25 (2007) 1627-1628; Kirkeby, A., et al., J. Neurosci. Methods 164 (2007) 50-58; Laugsch, M. et al., Int. J. Cancer 122 (2008) 1005-1011).

### SUMMARY OF THE INVENTION

The invention comprises an antibody binding to EPO-R which allows specific analysis of EPO-R especially in human tissue (e.g. biopsies or tissues).

The invention comprises an antibody binding to human EPO receptor, characterized in specifically binding human EPO receptor fragment LDKWLLPRNPPSEDLPGPGGSVDIV (SEQ ID NO:1), CSSALASKPSPEGASAASFEY (SEQ ID NO:2), or GGLSDGPYSNPYENSLIPAAEP (SEQ ID NO:3).

The antibody is preferably a monoclonal or polyclonal antibody.

Preferably the antibody according to the invention is characterized in comprising as heavy chain variable domain CDR3 region a CDR3 region of SEQ ID NO: 4 or 12.

Preferably the antibody is characterized in that the heavy chain variable domain comprises CDR3 region of SEQ ID NO: 4, a CDR2 region of SEQ ID NO:5 and a CDR1 region of SEQ ID NO:6 or CDR3 region of SEQ ID NO:12, a CDR2 region of SEQ ID NO:13 and a CDR1 region of SEQ ID NO:14.

Preferably the antibody is characterized in that the heavy chain variable domain comprises a CDR3 region of SEQ ID NO: 4, a CDR2 region of SEQ ID NO:5 and a CDR1 region of SEQ ID NO:6 and in that the light chain variable domain comprises a CDR3 region of SEQ ID NO: 7, a CDR2 region of SEQ ID NO:8 and a CDR1 region of SEQ ID NO:9.

Preferably the antibody is characterized in that the heavy chain variable domain comprises a CDR3 region of SEQ ID NO: 12, a CDR2 region of SEQ ID NO:13 and a CDR1 region of SEQ ID NO:14 and in that the light chain variable domain comprises a CDR3 region of SEQ ID NO: 15, a CDR2 region of SEQ ID NO:16 and a CDR1 region of SEQ ID NO:17.

Preferably the antibody is characterized in that the heavy chain variable domain comprises SEQ ID NO:10 or 18.

Preferably the antibody is characterized in that the heavy chain variable domain comprises SEQ ID NO:10 and the light chain variable domain comprises SEQ ID NO:11.

Preferably the antibody is characterized in that the heavy chain variable domain comprises SEQ ID NO:18 and the light chain variable domain comprises SEQ ID NO:19.

An antibody according to the invention binds specifically to EPO receptor in ELISA, Western Blot, immunocytochemistry assays and immunohistochemistry assays.

An antibody according to the invention specifically binds EPO receptor in UT7 cells which are expressing EPO receptor endogenously or recombinantly.

Preferably the antibody according to the invention is characterized in binding to EPO-R with a binding affinity of at least 10⁻⁸ M⁻¹ to 10⁻¹² M⁻¹.

It is further preferred that the antibody is of mouse, rabbit or human origin.

The invention further comprises the use of an antibody according to the invention to analyze cells bearing/expressing EPO receptor.

Preferably an antibody according to the invention is used to analyze EPO receptor in human tissue samples. Preferably such analysis is performed by Western Blot, immunocytochemistry or immunohistochemistry.

Such analysis can be performed qualitatively (e.g. to detect whether a cell comprises EPO receptor) or qualitatively (e.g. to detect EPO receptor expression).

### DETAILED DESCRIPTION OF THE INVENTION

The term “antibody” encompasses monoclonal and polyclonal antibodies and the various forms of antibody structures including but not being limited to whole antibodies and antibody fragments.

“Antibody fragments” comprises a portion of a full length antibody, preferably the variable domain thereof, or at least the antigen binding site thereof. Examples of antibody fragments include diabodies, single-chain antibody molecules, and multispecific antibodies formed from antibody fragments. scFv antibodies are, e.g., described in Houston, J. S., Methods in Enzymol. 203 (1991) 46-96. In addition, antibody fragments comprise single chain polypeptides having the characteristics of a V_{H} domain, namely being able to assemble together with a V_{L} domain, or of a V_{L} domain binding to EPO-R, namely being able to assemble together with a V_{H} domain to a functional antigen binding site and thereby providing an antibody with the properties of specifically binding to human EPO-R.

The term “specifically binding human EPO receptor fragment LDKWLLPRNPPSEDLPGPGGSVDIV (SEQ ID NO:1), CSSALASKPSPEGASAASFEY (SEQ ID NO:2), or GGLSDGPYSNPYENSLIPAAEP (SEQ ID NO:3)” as used herein means binding to such a fragment in ELISA at a S/N ratio of 10 or more at an antibody concentration of 0.1 μg/ml.

The term “antibody binding to EPO-R” as used herein means binding of the antibody to human EPO-R in a cellular binding assay measured by microscopy analysis using cells recombinantly expressing EPO-R in an amount of 100.000 to 500.000 receptors per cell (EPO-R expressing cells). Binding is found if the antibody causes an S/N (signal/noise) ratio of 400 (or more) at an antibody concentration of 0.1 μg/ml.

The term “binding of EPO to EPO receptor” as used herein means binding of EPO to human EPO-R in a cellular binding assay measured by microscopy analysis using EPO-R expressing cells. Binding is found if EPO causes an S/N (signal/noise) ratio of 400 or more at an EPO concentration of 0.1 μg/ml.

The term “no unspecific binding of an antibody according to the invention to a cellular compound” as used herein means that an antibody according to the invention does not bind to a cellular compound in a cellular binding assay measured by microscopy analysis using cells which do not express EPO-R. No binding is found if said compound causes an S/N (signal/noise) ratio of no more than 10 at an antibody concentration of 0.1 μg/ml.

Specific binding of an antibody to EPO-R is found, if the antibody causes an S/N (signal/noise) ratio of 400 at an antibody concentration of 0.1 μg/ml in a cellular binding assay measured by microscopy analysis using an EPO-R expressing cell and causes an S/N (signal/noise) ratio of no more than 10 at an antibody concentration of 0.1 μg/ml in said cellular binding assay measured by microscopy analysis using said cell in its status wherein said cell does not express EPO-R (1.000 receptors per cell or lower, e.g. 100 receptors or lower).

Immunofluorescence signals of microscopy analysis are quantified by measuring the region of overlap between positive and negative (control, noise signal) fluorescent samples morphometrically. A useful tool is the “Measuring Colocalization” Algorithm from MetaMorph® Imaging software (www.moleculardevices.com).

An antibody according to the invention does not inhibit binding of EPO to EPO receptor. An antibody according to the invention is able to determine EPO receptor specifically in human cell and tissue samples. Binding of an antibody according to the invention to Epo-R does not activate (phosphorylate) EPO-R.

The term “epitope” denotes a protein determinant capable of specifically binding to an antibody. Epitopes usually consist of chemically active surface groupings of molecules such as amino acids or sugar side chains and usually epitopes have specific three dimensional structural characteristics, as well as specific charge characteristics. Conformational and nonconformational epitopes are distinguished in that the binding to the former but not the latter is lost in the presence of denaturing solvents.

The invention further comprises the use of an antibody according to the invention for the detection of EPO-R in human cells, tissues or biopsies.

In another aspect, the present invention provides a diagnostic composition comprising an antibody according to the invention for the detection of EPO-R in human cells, tissues or biopsies.

### Description of the Sequences:

SEQ ID NO: 1 synthetic peptide
SEQ ID NO: 2 synthetic peptide
SEQ ID NO: 3 synthetic peptide
SEQ ID NO:4 Heavy chain CDR3 Clone 21.3.1
SEQ ID NO:5 Heavy chain CDR2 Clone 21.3.1
SEQ ID NO:6 Heavy chain CDR1 Clone 21.3.1
SEQ ID NO:7 Light chain CDR3 Clone 21.3.1
SEQ ID NO:8 Light chain CDR2 Clone 21.3.1
SEQ ID NO:9 Light chain CDR1 Clone 21.3.1
SEQ ID NO:10 Heavy chain Clone 21.3.1
SEQ ID NO:11 Light chain Clone 21.3.1
SEQ ID NO:12 Heavy chain CDR3 Clone 19.1.2
SEQ ID NO:13 Heavy chain CDR2 Clone 19.1.2
SEQ ID NO:14 Heavy chain CDR1 Clone 19.1.2
SEQ ID NO:15 Light chain CDR3 Clone 19.1.2
SEQ ID NO:16 Light chain CDR2 Clone 19.1.2
SEQ ID NO:17 Light chain CDR1 Clone 19.1.2
SEQ ID NO:18 Heavy chain Clone 19.1.2
SEQ ID NO:19 Light chain Clone 19.1.2

### DESCRIPTION OF THE FIGURES

FIG. 1 Specific binding of Mabs and Pabs to biotinylated EPOR peptides as determined by ELISA. Binding of PAK<EPOR(347-371)>K-IgG(IS)Ch01bSW to the biotinylated peptide 347-371 (corresponding to the mature EPOR) immobilized onto Maxisorp™ microtiter plates at 0.1 μg/ml. Mab Cl. 21.3.1 is not shown because this Mab is not suitable for ELISA under conditions used. Binding of Mabs Cl.19.1.2, Cl.19.3.7 and PAK<EPOR(382-402)>K-IgG(IS)Ch01bSW to the biotinylated peptide 382-402 (corresponding to the mature EPOR) immobilized onto Maxisorp™ microtiter plates. Binding of PAK<EPOR(454-475)>K-IgG(IS)Ch01bSW to the biotinylated peptide 454-475 (corresponding to the mature EPOR) immobilized onto Maxisorp™ microtiter plates.

FIG. 2 WB analysis of lysates from HELAwt, HELA-EPOR and UT-7 cells (to show specificity). (a) Specific binding of Mab Cl. 21.3.1 (epitope aa347-371) and PAK<EPOR(347-371)>K-IgG(IS)Ch01bSW. (b) Specific binding of Mab Cl. 19.3.7 and Mab Cl. 19.1.2 (epitope aa382-402) and PAK<EPOR(382-402)>K-IgG(IS)Ch01bSW. (c) Specific binding of PAK<EPOR(454-475)>K-IgG(IS)Ch01bSW (epitope aa 454-475)

FIG. 3 Immunocytochemistry analysis of HELAwt and HELA-EPOR. Double immunofluorescence of recombinant human EPOR-GFP (green) and antibody immunoreactivity (red). Specific labeling is indicated by colocalization of the red and green signal. HELAwt do not express EPOR and are used as negative control. (a) Staining with Mab Cl. 21.3.1 (aa 347-371), (b) Mab Cl.19.1.2 (aa382-402), (c) Mab Cl.19.3.7 (aa382-402).

FIG. 4 Immunohistochemistry analysis of HELAwt and HELA-EPOR and comparison to commercial antibody C-20 (SantaCruz). (a) polyclonal antibody C-20 from Santa-Cruz; (b) polyclonal affinity purified antibody PAK<EPOR(347-371)>K-IgG(IS)Ch01bSW.

FIG. 5 Comparative Western Blot analysis. Per lane 2.5×10⁴ cells were loaded. The antibody concentrations were: (A) PAK<EPOR(347-371) (10 ng/ml); (B) C-20 (0.4 μg/ml); (C) ABIN98954 (0.4 μg/ml); (D) M-20 (0.4 μg/ml); (E) ab10653 (0.4 μg/ml) and (F) BAF307 (0.4 μg/ml). Lanes, left to right: Hela parental (1), untreated (2), Opti-MEM® (3), non coding siRNA (4), EPO-R siRNA (5), EPO-R siRNA (6).

FIG. 6 Western Blot analysis of MAB307. Per lane total protein of 2.5×10⁴ cells were loaded and the primary antibody was used in a concentration of 0.4 μg/ml. Analysis was performed under denaturing (A) and native (B) conditions. Lanes, left to right: Hela parental (1), untreated (2), Opti-MEM® (3), non coding siRNA (4), EPO-R siRNA (5), EPO-R siRNA (6).

### EXAMPLE 1

### Generation of Monoclonal and Polyclonal Antibodies Directed Against the Intracellular Domain of the Human EPOR

Mab Cl. 21.3.1 and PAK<EPOR(347-371)>K-IgG(IS)Ch01bSW: a 25 amino-acid synthetic peptide corresponding to residues 347-371 of the mature human erythropoietin receptor (LDKWLLPRNPPSEDLPGPGGSVDIV; SEQ ID NO:1) was used as immunogen (corresponds to aa371-395 of the EPOR precursor).

Mab Cl.19.3.7, Mab Cl.19.1.2 and PAK<EPOR(382-402)>K-IgG(IS)Ch01bSW: a 21 amino-acid synthetic peptide corresponding to residues 382-402 of the mature human erythropoietin receptor (CSSALASKPSPEGASAASFEY; SEQ ID NO:2) was used as immunogen (corresponds to aa406-426 of the EPOR precursor).

PAK<EPOR(454-475)>K-IgG(IS)Ch01bSW: a 22 amino-acid synthetic peptide corresponding to residues 454-475 of the mature human erythropoietin receptor (GGLSDGPYSNPYENSLIPAAEP; SEQ ID NO:3) was used as immunogen (corresponds to aa478-499 of the EPOR precursor).

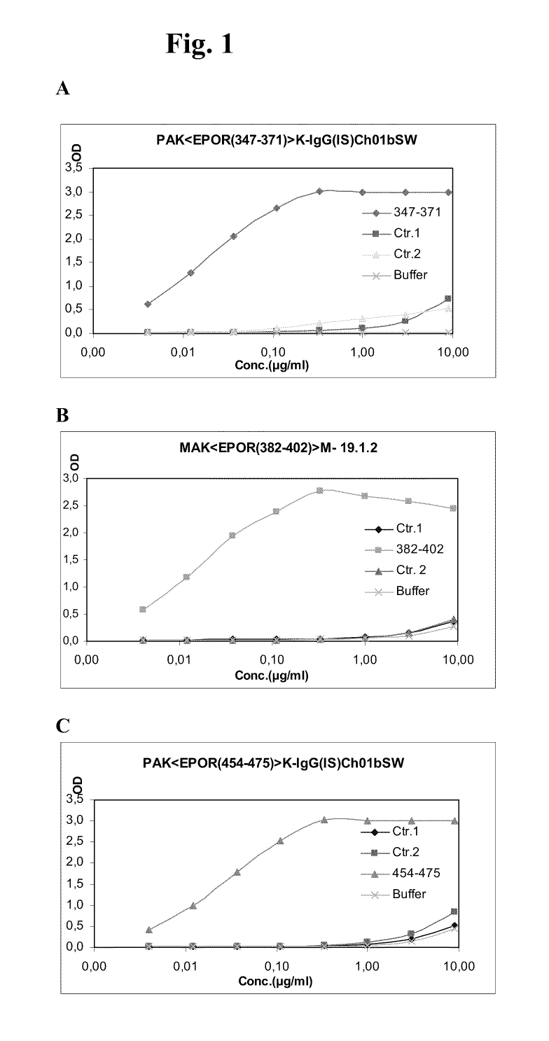

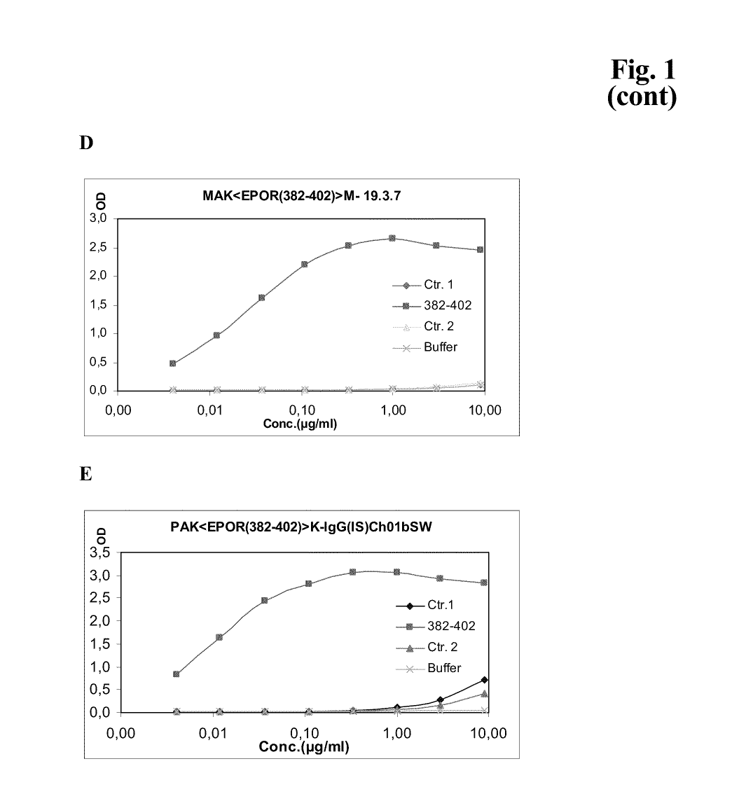

For immunization the peptides were coupled to KLH via a C terminal cystein. Rabbits and Balb/c mice were immunized with the protein every 4 weeks for 3-5 times. In addition, Balb/c mice received an i.v. boost on day 4 before fusion, splenocytes were harvested, and fused with Ag8 myeloma cells. Screening for specific antibodies was done by testing on protein coated ELISA microtiter plates (FIG. 1). Mab clones and polyclonal sera of rabbits were selected based on the detection of one specific band corresponding to the EPOR on Western Blots of cell lysates.

### EXAMPLE 2

### Generation of EPOR Over Expressing HELA Cells

For generating stably transfected HELA cells expressing recombinant EPOR, cells were transduced with the supernatant from GP2-293 cells (Clontech Laboratories, Inc) transiently transfected with a retroviral expression vector encoding EPOR or EPOR/EGFP (as fusion protein to the intracellular C-terminus, Invitrogen) and pVSV-G (an expression vector encoding the G glycoprotein of the rhabdovirus vesicular stomatitis virus). Two days after transduction the medium was replaced with fresh supplemented RPMI containing 0.2 mg/ml Zeocin™.

For transient transfection experiments 8×10⁴ HELA cells were plated on cover slips in a 12-well plate in 1 ml medium using FuGENE® Transfection reagent (Roche Molecular Biochemicals Cat. No. 1815075). In detail, 3 μl of FuGENE® 6 were added to 97 μl RPMI 1640 without FCS, incubated for 5 min at RT. Then, 1 μg DNA, mix was added, incubated for 15 min. at RT. Finally, 50 μl of the DNA/FuGENE® 6 solution was added to 1 ml cell culture medium containing the cells on cover slips.

### EXAMPLE 3

### Generation of EPOR Overexpressing UT7 Cells

UT-7 cell line is a human factor-dependent erythroleukemic cell line (Human bone marrow acute myeloid leukemia cell line DSMZ: ACC 137), requiring EPO for long-term growth. UT7 cells were maintained in RPMI medium supplemented with L-glutamine (2 mM), non-essential amino acids (1×), sodium pyruvate (1 mM), 10% fetal calf serum and 10 U/ml GM-CSF. Transduced cells (UT7/EPOR) were maintained in the same medium as non transduced cells (25 U/ml GM-CSF instead of 10 U/ml) with the addition of 0.4 mg/ml Zeocin™. Before each stimulation the cells were starved by incubation overnight in RPMI media supplemented with L-glutamine (2 mM), non-essential amino acids (1×), sodium pyruvate (1 mM) and 0.1% fetal calf serum.

UT-7 cells were transduced with the supernatant from GP2-293 (Clontech Laboratories, Inc) cells transiently transfected with a retroviral expression vector encoding EPO-R and pVSV-G (an expression vector encoding the G glycoprotein of the rhabdovirus vesicular stomatitis virus). Two days after transduction the medium was replaced with fresh supplemented RPMI containing 0.4 mg/ml Zeocin™ and 25 U/ml GM-CSF. After selection a cell line of UT-7 cells stable expressing EPOR on their surface was obtained.

### EXAMPLE 4

### Immunoprecipitation

UT7 cells were lysated in ice-cold lysis buffer [Tris 20 mM (pH7.4), NaCl 137 mM, Glycerol 10%, Nonidet P-40 1%, protease inhibitors 1× (Pierce, # 78410), phosphatase inhibitors 1× (Pierce #78420)] for 30 minutes at 4° C. followed by centrifugation at 13000 rpm for 10 minutes at 4° C. (Eppendorf centrifuge). The precleared lysate supernatants was incubated overnight at 4° C. with the antibody MAB307 (mouse monoclonal anti-human EPO-R extracellular domain, R&D Systems) and Protein G agarose beads. The beads were washed three times in lysis buffer and heat for 10 minutes at 70° C. in NuPAGE® sample buffer (Invitrogen) in reducing conditions.

### EXAMPLE 5

### SDS-PAGE and Western Blotting

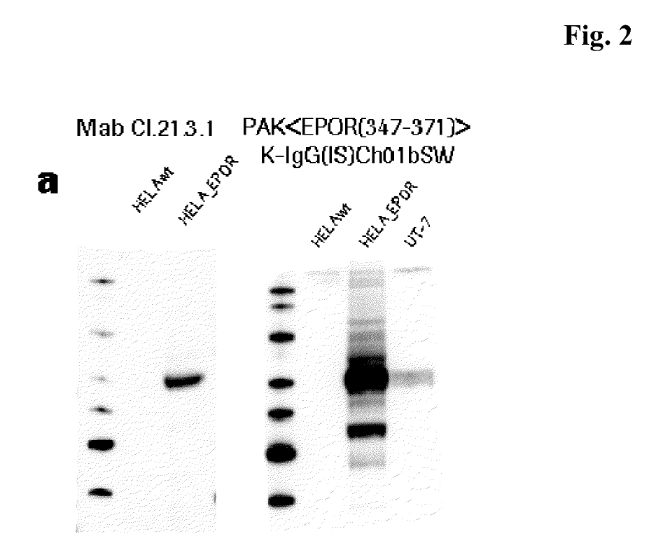

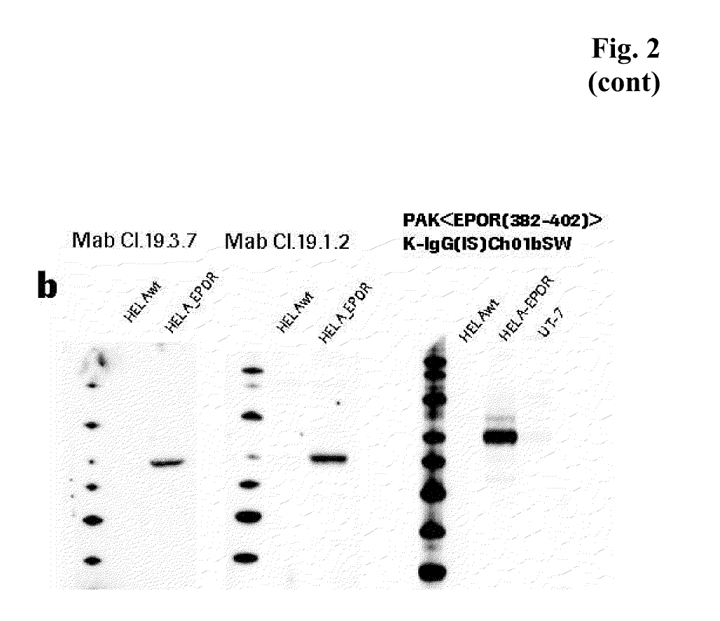

The SDS-PAGE and western blotting were performed according to standard procedures and the NuPAGE® gel system of Invitrogen. The extracts corresponding to different number of cells were loaded in each line of a NuPAGE® Novex® 4-12% Bis-Tris gel. The proteins were then transferred onto PVDF membranes and incubated with the respective antibodies overnight at 4° C. After washing, the membranes were incubated with a conjugate anti-mouse or anti-rabbit IgG-POD and developed using ECL reagents (Lumi-Light^{PLUS} western blotting substrate, Roche Diagnostics GmbH): Results are shown in FIG. 2.

### EXAMPLE 6

### BIACORE™ Analysis

Measurements were made on a BIACORE™ 3000 at 25° C. in HBS-EP-Buffer, pH 7,4 (10 mM HEPES, 150 mM NaCl, 3.4 mM EDTA, 0.005% polysorbate 20 (w/v). 1.0 mg/ml CMD was added to reduce unspecific binding. Results are shown in Table 1.

Table 1: Determination of binding affinity/avidity by BIACORE™ analysis. Avidity as determined by binding to immobilized biotinylated peptides. All antibodies (except for 21.3.1) display nano-/subnanomolar avidity to their corresponding EPOR peptide.

TABLE 1

| Peptide | Antibody | 1/Ms | 1/sec | Min | 1/M | nM |
|---|---|---|---|---|---|---|
| 382-402 | Mab 19.3.7 | 1.7 × 10⁶ | 2.2 × 10⁻³ | 5 | 9.9 × 10⁸ | 1 |
| | Mab 19.1.2 | 1.9 × 10⁶ | 2.1 × 10⁻³ | 5 | 1.1 × 10⁹ | 1 |
| 347-371 | Polyclonal | 2.9 × 10⁵ | 1.1 × 10⁻⁴ | 109 | 2.8 × 10⁹ | 0.4 |
| 382-402 | Polyclonal | 3.1 × 10⁶ | 1.0 × 10⁻⁴ | 117 | 3.5 × 10⁹ | 0.03 |
| 454-475 | Polyclonal | 2.6 × 10⁵ | 2.0 × 10⁻⁴ | 57 | 1.3 × 10⁹ | 0.8 |

### EXAMPLE 7

### Immunocytochemistry and Immunohistochemistry

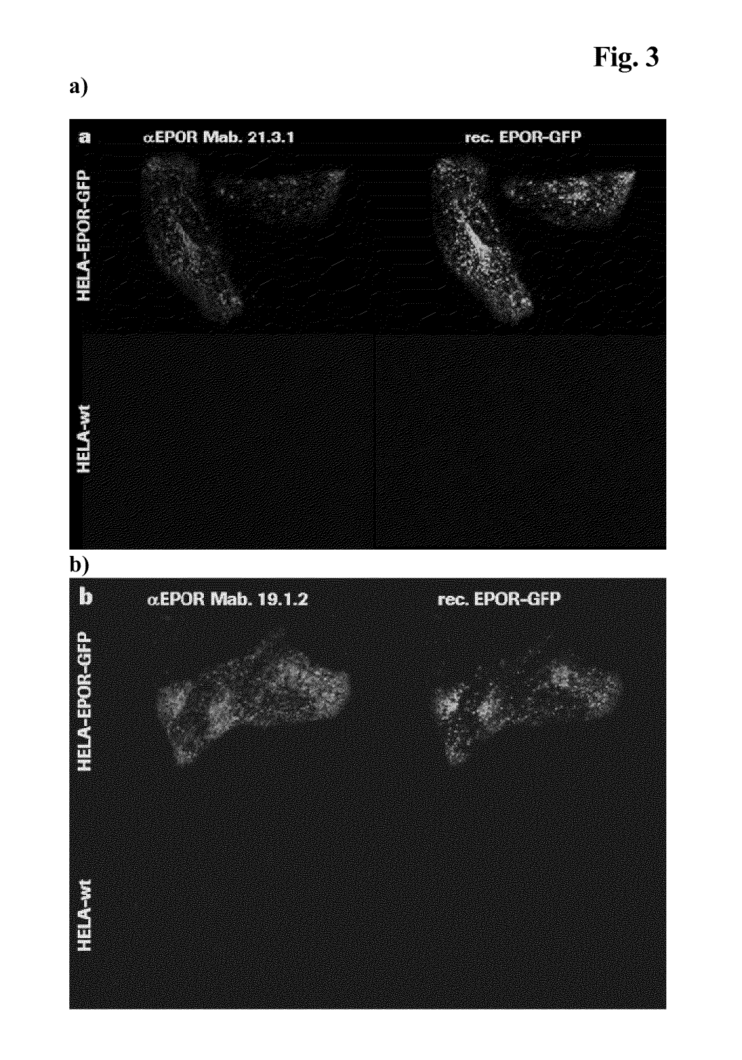

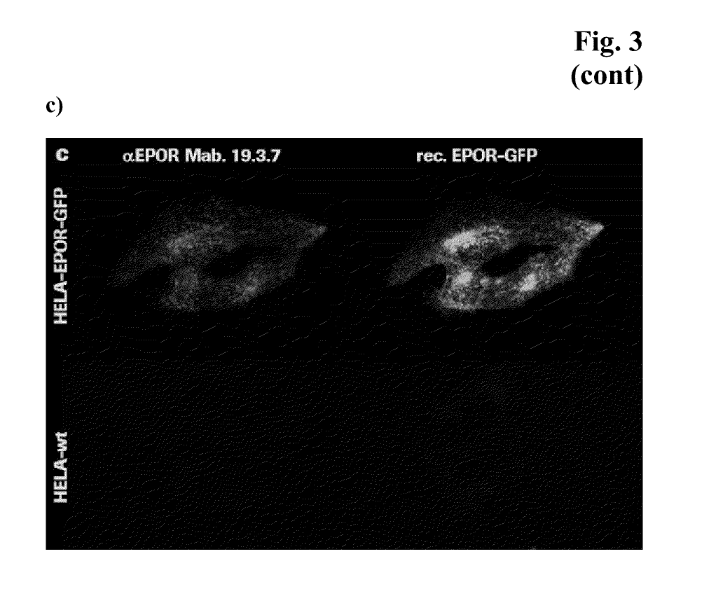

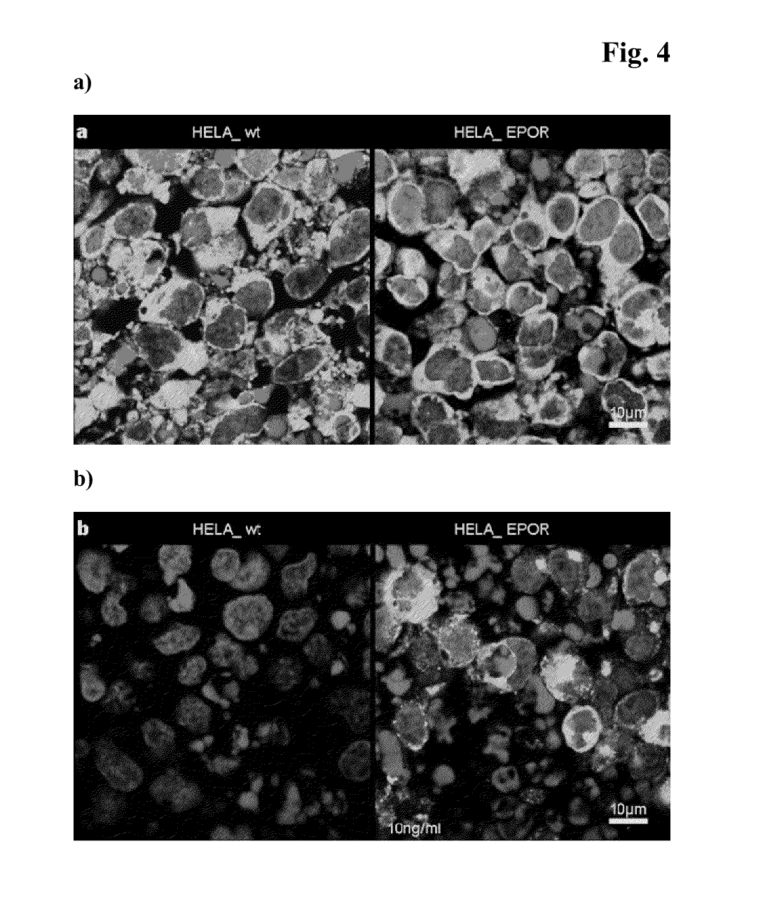

For immunofluorescence studies, cells were grown on glass coverslips (170 μm thickness) in RPMI1640, 10% FCS until 80% confluency. Cultures were incubated with antibody samples @ 10 μg/ml for 45 min, washed and fixed with 4% PFA. Bound antibodies were detected by Alexa Fluor® 488 goat anti-human IgG secondary antibodies. Specimens were imaged on a LEICA confocal laser scanning microscope SP2 using 488 nm and 633 nm excitation for Alexa Fluor® 488 and AlexaFluor® 633 respectively. Results are shown in FIGS. 3 and 4.

Immunocytochemistry analysis of affinity purified polyclonal antibodies PAK<EPOR(347-371)>K-IgG(IS)Ch01bSW (A) and PAK<EPOR(454-475)>K-IgG(IS)Ch01bSW(B) directed against EPOR on transiently transfected HELA_EPOR cells were performed as follows: HELA cells cultured on glass coverslips were transfected to transiently express EPOR-GFP, PFA, fixed and stained w/ 1.0 μg/ml purified IgG of PAK<EPOR(347-371)>K-IgG(IS)Ch01bSW directed against EPOR. Anti-EPOR antibody immunoreactivity was found to be closely colocalized with the green fluorescent rec. EPOR. The antibody also recognizes newly synthesized EPOR that is confined to the ER/Golgi region. The lack of any detectable labeling in non-transfected cells also confirms the high specificity of the anti-EPOR antibodies PAK<EPOR(347-371)>K-IgG(IS)Ch01bSW and PAK<EPOR(454-475)>K-IgG(IS)Ch01bSW.

### EXAMPLE 8

### Quantitative Evaluation of Specificity of PAK<EPOR(347-371)>K-IgG(IS)Ch01bSW

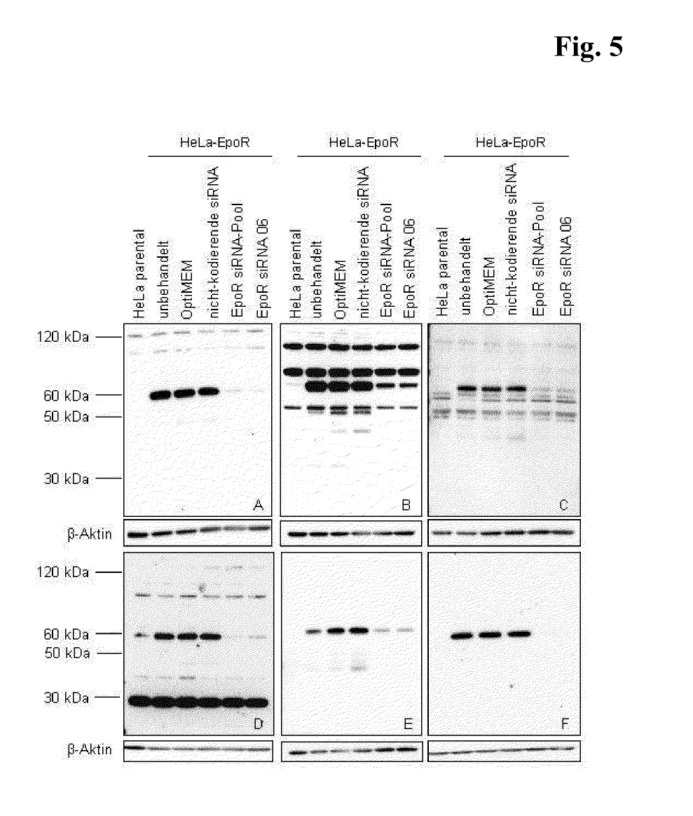

HELA cells cultured on glass coverslips were transfected to transiently express EPOR-GFP, PFA, fixed and stained w/ 1.0 μg/ml purified IgG of PAK<EPOR(347-371)>K-IgG(IS)Ch01bSW directed against EPOR. Note the close colocalization of the anti-EPOR antibody immunoreactivity with the green fluorescent rec. EPOR. The antibody also recognizes newly synthesized EPOR that is confined to the ER/Golgi region. The lack of any detectable labelling in non-transfected cells also shown in the field (as indicated by the blue cell nuclei labelled with DAPI) confirms the high specificity of the anti-EPOR antibodies PAK<EPOR(347-371)>K-IgG(IS)Ch01bSW and PAK<EPOR(454-475)>K-IgG(IS)Ch01bSW. Percentage of overlap of PAK<EPOR(347-371)>K-IgG(IS)Ch01bSW immunoreactivity with fluorescence of recombinant EPOR-GFP is determined as >97% using the “Measuring Colocalization” Algorithm from MetaMorph® Imaging software (FIG. 5).

### EXAMPLE 9

### Comparison with Commercial Available Antibodies Against EPOR

The antibodies of Table 2 were investigated:

TABLE 2

| Name | Origin | Isotyp | Supplier |
|---|---|---|---|
| ABIN166173 | mouse monoclonal | IgG2b Kappa | Abnova GmbH |
| ABIN170186 | mouse polyclonal | IgG | Abnova GmbH |
| ABIN98954 | sheep polyclonal | IgG | Abnova GmbH |
| BAF307 | goat polyclonal | IgG | R&D Systems |
| | | | GmbH |
| MAB307 | mouse monoclonal | IgG2b | R&D Systems |
| | | | GmbH |
| H-194 | rabbit polyclonal | IgG | Santa Cruz Inc. |
| C-20 | rabbit polyclonal | IgG | Santa Cruz Inc. |
| M-20 | rabbit polyclonal | IgG | Santa Cruz Inc. |
| Ww-12 | mouse monoclonal | IgG2b | Santa Cruz Inc. |
| PA1-20180 | goat polyclonal | IgG | Dianova GmbH |
| ab10653 | goat polyclonal | IgG | Abcam plc |
| ab54659 | mouse monoclonal | IgG2b Kappa | Abcam plc |
| ab56310 | mouse monoclonal | IgG2b Kappa | Abcam plc |

FIG. 8 shows that five commercial antibodies detect EPOR at a size of about 60 kD based on EPO-R RNA interference (C-20, ABIN98954, M-20, ab10653 und BAF307). At similar EPOR band intensities, four antibodies (C-20, ABIN98954, ab10653 und M-20) detect besides a 60 kDa band additional Western Blot bands in the tumor cell lines. Four antibodies do not detect protein bands with a molecular weight of about 60 kD that are affected by EPOR siRNA (H-194, ab54659, ab56310, ABIN170186 and ABIN166173). Antibodies Ww-12 and PA1-20180 did not show any detectable chemiluminescence signal at an antibody concentration of 0.4 μg/ml despite the presence of IgG.

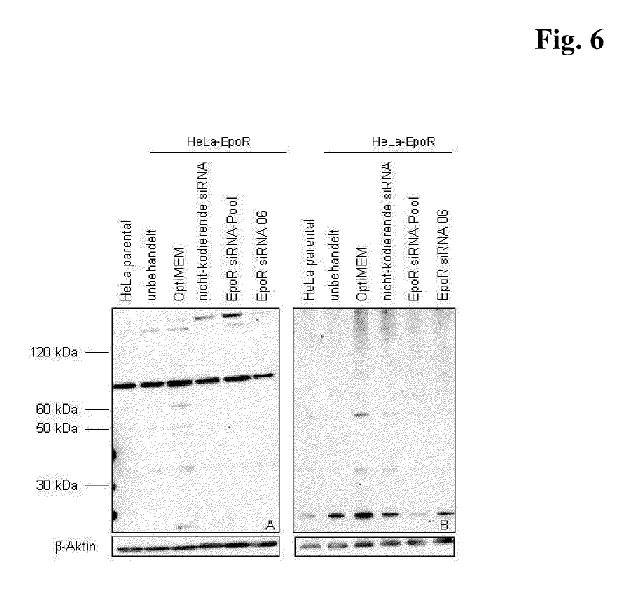

FIG. 6 shows that MAB307 did not provide a 60 kDa band under denaturing or native conditions in HeLa und HeLa-EpoR cell lysates. The antibody detects under detaturing conditions an unspecific protein at about 80 kDa. In native samples the antibody recognizes a 20 kDa protein.

Antibody C-20 shows in addition a significant cross reactivity with Hsp70 protein. Using a Western Blot assay (total protein from 2.5×10⁴ cells and 10 ng/ml antibody, in contrast to all tested commercial available antibodies tested, PAK<EPOR(347-371)> detects specifically a prominent EPOR specific band of about 60 kDa.

For the investigation of the sensitivity of Western blot assay using the various EPOR antibodies a matrix was established of HeLa-EpoR cells in descending cell numbers which was supplemented with parental HeLa cells up to a total cell number of 1×10⁵ cells per lane. Decrease of cell numbers occurs in steps of 1×10⁵, 3×10⁴, 1×10⁴, 3×10³, 1×10³ and 0 HeLa-EpoR cells per lane. Antibody concentration was 0.4 μg/ml and light exposed for 1.5 min (Lumi Imager™) Results are shown in Table 3.

TABLE 3

| | Detection limit [number of |
|---|---|
| Name | HeLa-EPOR cells] |
| ABIN98954 | 1 × 10⁴ |
| BAF307 | 3 × 10⁴ |
| C-20 | betweenn 1 × 10⁴ and 3 × 10³ |
| M-20 | 3 × 10⁴ |
| ab10653 | 3 × 10³ |
| PAK < EPOR(347-371) | 1 × 10³ |

1. An antibody binding to human erythropoietin (EPO) receptor, said antibody specifically binding human EPO receptor fragment LDKWLLPRNPPSEDLPGPGGSVDIV (SEQ ID NO:1), CSSALASKPSPEGASAASFEY (SEQ ID NO:2), or GGLSDGPYSNPYENSLIPAAEP (SEQ ID NO:3).

2. The antibody according to claim 1, wherein the heavy chain variable domain of said antibody comprises a CDR3 region of SEQ ID NO: 4 or 12.

3. The antibody according to claim 2, wherein the heavy chain variable domain comprises a CDR3 region of SEQ ID NO: 4, a CDR2 region of SEQ ID NO:5 and a CDR1 region of SEQ ID NO:6 or a CDR3 region of SEQ ID NO:12, a CDR2 region of SEQ ID NO:13 and a CDR1 region of SEQ ID NO:14.

4. The antibody according to claim 3, wherein the heavy chain variable domain comprises a CDR3 region of SEQ ID NO: 4, a CDR2 region of SEQ ID NO:5 and a CDR1 region of SEQ ID NO:6 and the light chain variable domain of said antibody comprises a CDR3 region of SEQ ID NO: 7, a CDR2 region of SEQ ID NO:8 and a CDR1 region of SEQ ID NO:9.

5. The antibody according to claim 3, wherein the heavy chain variable domain comprises a CDR3 region of SEQ ID NO: 12, a CDR2 region of SEQ ID NO:13 and a CDR1 region of SEQ ID NO:14 and in that the light chain variable domain comprises a CDR3 region of SEQ ID NO: 15, a CDR2 region of SEQ ID NO:16 and a CDR1 region of SEQ ID NO:17.

6. The antibody according to claim 1, wherein the heavy chain variable domain of said antibody comprises SEQ ID NO:10 or 18.

7. The antibody according to claim 1, wherein the heavy chain variable domain of said antibody comprises SEQ ID NO:10 and the light chain variable domain comprises SEQ ID NO:11.

8. The antibody according to claim 1, wherein the heavy chain variable domain comprises SEQ ID NO:18 and the light chain variable domain comprises SEQ ID NO:19.

9. A diagnostic kit comprising the antibody according to any one of claims 1 to 8.

10. A method for analyzing EPO receptor in a human tissue sample, comprising combining the antibody according to any one of claims 1 to 8 with the sample and analyzing binding of the antibody to EPO receptor.

11. The method according to claim 10, wherein the human tissue sample is a lysate of human tissue.

12. The method according to claim 10, wherein the analysis is performed by immunochemistry or immunohistochemistry analysis.

13. The method according to claim 11, wherein the analysis is performed by immunochemistry or immunohistochemistry analysis.

14. The method according to claim 10, wherein the analysis is performed by Western Blot.

15. The method according to claim 11, wherein the analysis is performed by Western Blot.
